(19) United States
(12) Patent Application Publication
(10) Pub. No.: US 2019/0038641 A1
(43) Pub. Date: Feb. 7, 2019

(54) COMBINATION THERAPY FOR CASTRATION-RESISTANT PROSTRATE CANCER

(71) Applicants: Bandana CHATTERJEE, San Antonio, TX (US); Sulgi PARK, San Antonio, TX (US); Chung S. SONG, San Antonio, TX (US)

(72) Inventors: Bandana CHATTERJEE, San Antonio, TX (US); Sulgi PARK, San Antonio, TX (US); Chung S. SONG, San Antonio, TX (US)

(73) Assignee: The Board of Regents of the University of Texas System, Austin, TX (US)

(21) Appl. No.: 16/075,355

(22) PCT Filed: Feb. 3, 2017
(86) PCT No.: PCT/US2017/016428

§ 371 Date: Aug. 3, 2018

Related U.S. Application Data
(60) Provisional application No. 62/291,187, filed on Feb. 4, 2016.

(51) Int. Cl. A61K 31/593; A61P 35/00

(52) U.S. Cl. CPC A61K 31/593; A61K 31/568; A61K 2300/00; A61P 35/00

(57) ABSTRACT

Certain embodiments of the current disclosure are directed to a combined vitamin D (calcitriol or analog thereof) and androgen therapy that reduces the growth of mCRPC in humans. In certain aspects a new combined vitamin D and androgen medication formulation is described that controls advanced prostate cancer. Calcitriol, calcitriol analogs, and androgens are referred to herein as therapeutic agents or agents.

### PRIORITY CLAIM

This Application claims priority to U.S. Provisional Patent Application Ser. No. 62/291,187 filed Feb. 4, 2016, which is incorporated herein by reference in its entirety.

### STATEMENT REGARDING FEDERALLY FUNDED RESEARCH

This invention was made with government support under W81XWH-14-1-0606 and 2I01BX000280-05A1 awarded by the Department of Defense and Department of Veterans Affairs. The government has certain rights in the invention.

### BACKGROUND

Prostate cancer (PC) is the most common malignancy in males and the second leading cause of cancer-related deaths in the United States and Europe. The frequency of PC has been increasing significantly in most developed countries probably due to prevalent western-style life-style and the explosion of the aging population (Gronberg, Lancet 2003, 361:859-64; Hsing and Devesa, Epidemiol Rev 2001, 23:3-13). Surgical and radiation therapies are effective to the localized disease, but nearly 30% of treated PC patients still suffer from the relapse of the disease (Feldman and Feldman, Nat Rev Cancer 2001, 1:34-45; Scher and Sawyers, J Clin Oncol 2006, 23:8253-61; Han et al., J Urol 2001, 166:416-9). Most of the patients with relapsed or advanced disease respond well to androgen-ablation therapy (medical or surgical castration) because PCs are usually androgen-dependent at a relatively early stage. However, they often acquire castration-resistant phenotype that progresses aggressively and ultimately leads to the death of PC patients. Both castration-naive prostate cancers (CNPCs) with high Gleason score, and castration-resistant prostate cancers (CRPCs) respond poorly to androgen-ablation therapy and have highly aggressive behavior and thus are associated with poor prognosis. Hence, development of new therapies based on the molecular mechanisms of prostate carcinogenesis or castration-resistant PC(CRPC) is needed.

### SUMMARY

Interference of the androgen-regulated androgen receptor axis with androgen deprivation therapy (ADT), which depletes serum androgens to a castrate level, is the standard first-line treatment for early-stage metastatic prostate cancer. Recurrence to a terminal stage develops by 18 to 24 months for˜70% patients who are on ADT, although androgen receptor activity persists in metastatic castration-resistant prostate cancer (mCRPC). Intratumoral androgen biosynthesis plays a key role in restoring androgen receptor signaling in mCRPC.

Certain embodiments of the current disclosure are directed to a combined vitamin D (calcitriol or analog thereof) and androgen therapy that reduces the growth of mCRPC in humans. In certain aspects a new combined vitamin D and androgen medication formulation is described that controls advanced prostate cancer. Calcitriol, calcitriol analogs, and androgens are referred to herein as therapeutic agents or agents.

In certain embodiments the calcitriol or analog thereof is independently formulated relative to the androgen, thus having two independent compositions to be administered in various regime relative to one another. In other embodiments the calcitriol or analog thereof and androgen (i.e., therapeutic agents) are formulated in the same composition and as such are administered simultaneously.

For the compounds of the present invention, alone or as part of a pharmaceutical composition, such doses are between about 0.001 mg/kg and 100 mg/kg body weight, between about 1 and 100 μg/kg body weight, or between 1 and 10 μg/kg body weight—including all values and ranges there between.

Therapeutically effective doses will be easily determined by one of skill in the art and will depend on the severity and course of the disease, the patient's health and response to treatment, the patient's age, weight, height, sex, previous medical history and the judgment of the treating physician.

In some methods of the invention, the cancer cell is a tumor cell. The cancer cell may be in a patient. The patient may have a solid tumor. In such cases, embodiments may further involve performing surgery on the patient, such as by resecting all or part of the tumor. Compositions may be administered to the patient before, after, or at the same time as surgery. In additional embodiments, patients may also be administered a therapeutic agent directly, endoscopically, intratracheally, intratumorally, intravenously, intralesionally, intramuscularly, intraperitoneally, regionally, percutaneously, topically, intrarterially, intravesically, or subcutaneously. Therapeutic compositions may be administered 1, 2, 3, 4, 5, 6, 7, 8, 9, 10, 11, 12, 13, 14, 15, 16, 17, 18, 19, 20 or more times, and they may be administered every 1, 2, 3, 4, 5, 6, 7, 8, 9, 10, 11, 12, 13, 14, 15, 16, 17, 18, 19, 20, 21, 22, 23, 24 hours, or 1, 2, 3, 4, 5, 6, 7 days, or 1, 2, 3, 4, 5 weeks, or 1, 2, 3, 4, 5, 6, 7, 8, 9, 10, 11, 12 months. In certain aspects the therapeutic agents can be administer within 1, 2, 3, 4, 5, 6, 7, 8, 9, 10, 20, 30, 40, 50, 60 or more minutes, hours or days of each other. In other aspects the therapeutic agents are administered simultaneously or within a few seconds of each other. In still further embodiments the subject or patient can be treated using other known prostate cancer therapies, such as surgery, radiation, or chemotherapy.

The term “combination therapy”, as used herein, refers to those situations in which two or more different pharmaceutical agents are administered in overlapping regimens so that the subject is simultaneously exposed to both agents.

A “dosing regimen”, as that term is used herein, refers to a set of unit doses (typically more than one) that are administered individually separated by periods of time. The recommended set of doses (i.e., amounts, timing, route of administration, etc.) for a particular pharmaceutical agent constitutes its dosing regimen.

The term “castration-resistant prostate cancer (CRPC)” refers to cancers that are tolerant to androgen-ablation therapy (castration). As used herein, the term “CRPC” includes androgen-independent phenotype that has been termed hormone-refractory prostate cancers (HRPCs).

The term “effective amount” means that amount of a drug(s) or pharmaceutical agent that will elicit the biological or medical response of a tissue, cell, system, animal, or human that is being sought by a researcher or clinician. The term “therapeutically effective amount” means any amount which, as compared to a corresponding subject who has not received such amount, results in improved treatment, healing, prevention, or amelioration of a disease or disorder, or a decrease in the rate of advancement of a disease or disorder, and also includes amounts effective to enhance normal physiological function. In certain aspects the disease is prostate cancer.

The terms “treat” or “treatment” as applied to cancer refer to partially or totally inhibiting, delaying, or preventing the progression of cancer including cancer metastasis; inhibiting, delaying, or preventing the recurrence of the cancer including cancer metastasis in a mammal, including humans.

The terms “patient” and “subject” as used herein refers to any mammal, and more preferably humans.

The phrase “other agents or therapies” is meant to describe additional medicinal compounds or treatments that are administered in treating a prostate cancer patient. Typical treatments for cancer involve chemotherapy and/or bone marrow transplantation and/or radiation therapy. Other types of therapies include radiation therapy, which involves the use of high energy rays.

The phrase “pharmaceutically acceptable carrier” as used herein means a pharmaceutically acceptable material, composition or vehicle, such as a liquid or solid filler, diluent, excipient, solvent or encapsulating material useful for formulating a drug for medicinal or therapeutic use. Each carrier must be “acceptable” in the sense of being compatible with other ingredients of the formulation and not unacceptably injurious to the patient.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Regulation of HSD3β-1 in C4-2B castration-resistant cell lines.

FIG. 2. Regulation of CYP24A1 in C4-2B castration resistant cell lines.

FIG. 3. QRT-PCR assay of mRNAs for CYP24A1 (A), SULT2B1 (B), CYP3A4 (C) in C4-2B human castration-resistant prostate cancer cells treated with vehicle, EB1089 (10 nM), EB 1089 (10 nM)+R1881 (1 nM), or R1881 (1 nM).

### DESCRIPTION

One strategy for treating prostate cancer (PC) is androgen-ablation therapy (medical or surgical castration) because PCs are usually androgen-dependent at a relatively early stage. Androgens bind to a specific receptor, the androgen receptor (AR), inside the cells of target tissues. The AR is expressed in numerous tissues of the body and is the receptor through which the physiological as well as the pathophysiological effects of endogenous androgen ligands, such as testosterone (T) and dihydrotestosterone (DHT), are expressed. A compound that binds to the AR and mimics the effects of an endogenous AR ligand is referred to as an AR agonist, whereas a compound that inhibits the effects of an endogenous AR ligand is termed an AR antagonist. Binding of androgen to the receptor activates it and causes it to bind to DNA binding sites adjacent to target genes. From there it interacts with coactivator proteins and basic transcription factors to regulate the expression of the gene. Thus, via its receptor, androgens cause changes in gene expression in cells. These changes ultimately have consequences on the metabolic output, differentiation or proliferation of the cell that are visible in the physiology of the target tissue. In the prostate, androgens stimulate the growth of prostate tissue and prostate cancer cells by binding to the AR that is present within the cytoplasm of androgen sensitive tissue.

Agents that block the action (antiandrogens) of endogenous hormones (e.g., testosterone or 5α-dihydrotestosterone) are highly effective and routinely used for the treatment of prostate cancer (androgen ablation therapy). While initially effective at suppressing tumor growth, these androgen ablation therapies eventually fail in almost all subjects, leading to “castration resistant prostate cancer” (“CRPC”). Most, but not all, prostate cancer cells initially respond to androgen withdrawal therapy. However, with time, surviving populations of prostate cancer cells emerge because they have responded to the selective pressure created by androgen ablation therapy and are now refractory to it. Not only is the primary cancer refractory to available therapies, but cancer cells may also break away from the primary tumor and travel in the bloodstream, spreading the disease to distant sites (especially bone). Among other effects, this causes significant pain and further bone fragility.

It is contemplated that CRPC cells survive in an environment characterized by low levels of circulating androgens by amplifying at least three different pathways to enhance the response to the intracellular androgens that remain available. These include: (1) Up-regulation of the expression of the AR, which increases AR copy number and hence the sensitivity of the cells to low levels of circulating androgen induced by medical castration therapy; (2) Increase in the expression of enzymes involved in the importation of androgens that remain in cells after androgen deprivation therapy; (3) Increase in the expression of genes that regulate steroidogenesis, permitting the CRPC cells to synthesize their own androgens.

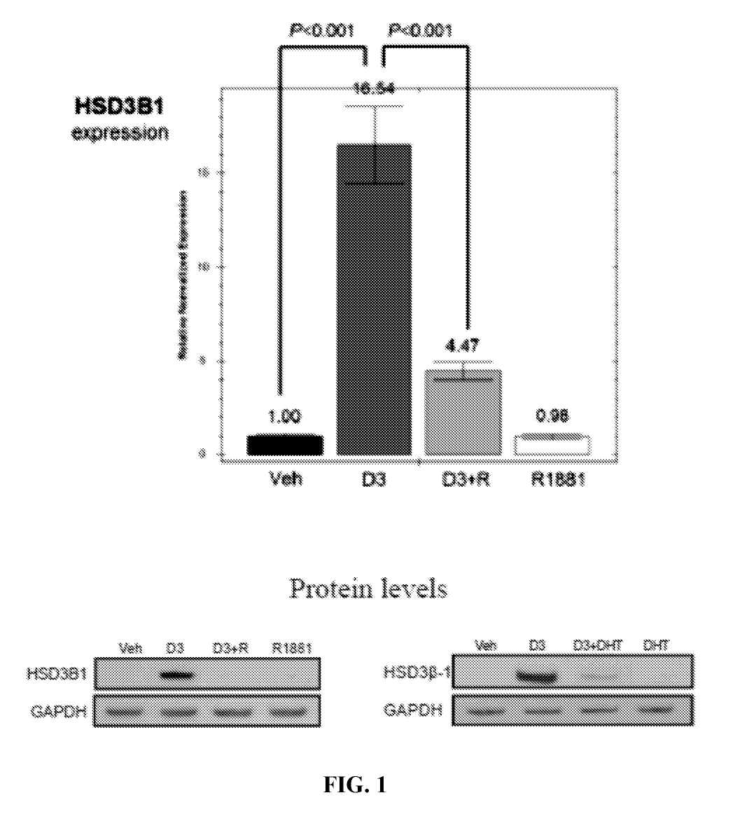

The studies described herein show in a prostate cancer cell culture model of castration-resistant prostate cancer that 3-β hydroxysteroid dehydrogenase-type I (HSD3β-1), which is a key enzyme for driving androgen biosynthesis both normally and in the tumor tissue of castration-resistant prostate cancer in humans, is strongly induced by calcitriol and this induction is completely lost when the cells were treated simultaneously with calcitriol and an androgen (either R1881, a synthetic androgen or 5α-dihydrotestosterone (DHT), a natural androgen in the body). Androgen alone did not cause any significant changes in the expression of HSD3β-1 (FIG. 1). Induction of HSD3β-1 was observed at messenger RNA levels (quantitative RT-PCR assay) and protein levels (Western blot assay) and the changes were observed in two different castration-resistant prostate cancer cell lines (C4-2B and C4-2).

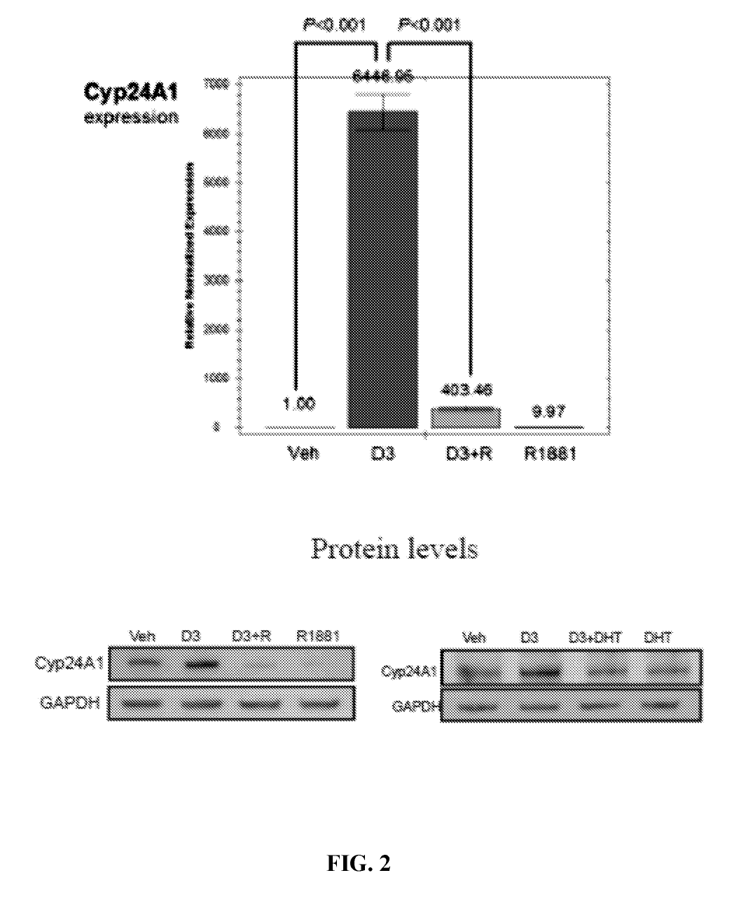

For the enzyme CYP24A1, which metabolizes calcitriol to the biologically inactive metabolite 1,24,25 (OH)₃D₃, similar regulations were observed—i.e., highly robust induction of CYP24A1 by calcitriol (by three orders of magnitude and more) and complete loss of this induction in the presence of androgen (FIG. 2). CYP24A1 induction by calcitriol has been reported by many laboratories. However, complete suppression of this induction by androgen has not been reported in the literature.

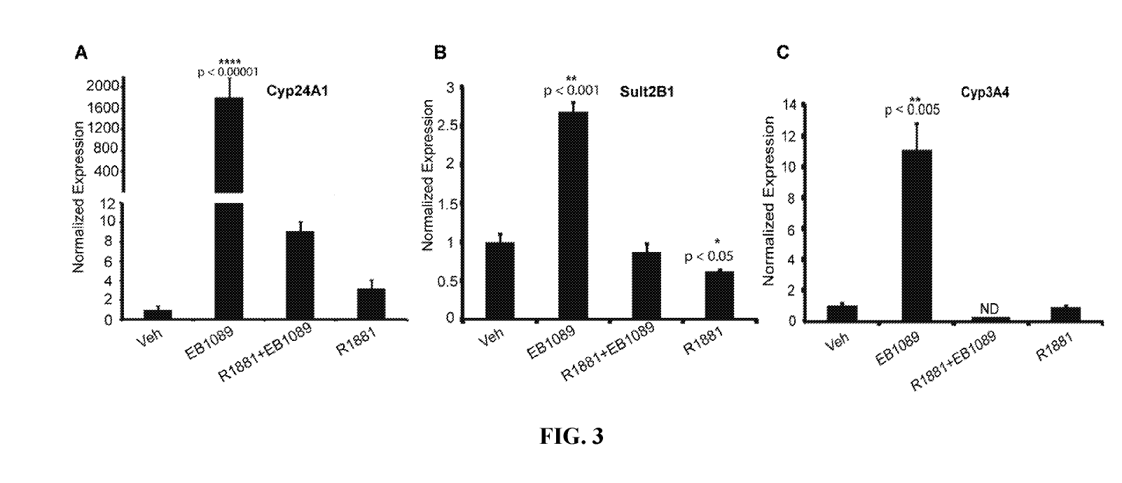

Treatment of C4-2B castration-resistant cells concurrently with R1881 (a synthetic androgen) and EB1089 (a calcitriol analog) completely abrogated the induction of SULT2B1 mRNAs observed with EB1089 alone (FIG. 3B). Similarly, EB1089 induced CYP3A4 mRNA expression by more than 10-fold, while co-treatment with R1881 drastically reduced the level of CYP3A4 mRNAs, bringing it to the basal level or even lower (FIG. 3C). EB1089-induced robust expression of CYP24A1 (˜2000-fold) was also drastically reduced (to less than 10-fold) by the combined action of R1881 and EB1089 (FIG. 3A). Dampened vitamin D/VDR-induced CYP24A1 mRNA expression in the presence of androgen is beneficial, as it would prevent ligand degradation, thereby enhancing VDR signaling.

Calcitriol, also called 1,25-dihydroxycholecalciferol or 1,25-dihydroxyvitamin D3, is the hormonally active metabolite of vitamin D with three hydroxyl groups (abbreviated 1,25-(OH)2D3 or simply 1,25(OH)2D). Calcitriol increases the level of calcium (Ca2+) in the blood by increasing the uptake of calcium from the gut into the blood, and possibly increasing the release of calcium into the blood from bone. Calcitriol is prescribed for the treatment of hypocalcaemia, osteoporosis, and the prevention of corticosteroid-induced osteoporosis.

Calcitriol is well known for its inhibition of prostate cancer in experimental models—both in cell culture and in xenograft tumors in immune-deficient mice. However, human trials with vitamin D therapy for inhibiting prostate cancer have not been successful, partly due to the fact that high doses of calcitriol required to inhibit prostate cancer in human patients are highly toxic due to vitamin D's hypercalcemic effect.

Intra-tumor androgen production plays a key role in the progression of therapy-resistant prostate cancer. However, high-doses of androgen administration can cause inhibition of therapy-resistant prostate cancer in animal models in a xenograft tumor setting and in cell culture models. Furthermore, it has been reported that high levels of CYP24A1 interfere with the anti-cancer effects of vitamin D.

The results described herein signify that combination therapy of vitamin D (or other vitamin D analogs) at a nontoxic dose with androgen can potentially lower intra-tumor androgen production by reducing the expression and activity of HSD3β-1. Vitamin D refers to molecules that serve as prohormones for the bioactive hormone calcitriol. In certain aspects calcitriol analogs are administered as part of the combination therapy described herein. Calcitriol analogs can include, but is not limited to 1α(OH)D₂, 1α(OH)D₅, LG190119, OCT, EB1089, TX522, TX527, 19-nor-1α, 25(OH)₂D₂, Ro25-9022, 16-ene-23-yne, Ro23-7553, Ro24-5531, Ro25-6760, Ro-4383561, or JK1626-2 (See Masuda and Jones, Molecular Cancer Therapeutics, 2006 5:797). A number of vitamin D and calcitriol analogs are known in the art. At the same time, suppression of CYP24A1 by androgen administration will protect calcitriol from CYP24A1-mediated degradation, so that vitamin D will remain active at a low, non-toxic dose. Furthermore, androgen administration is expected to prevent the inhibitory effect of CYP24A1 on vitamin D's anti-cancer effects. An androgen can be a natural or synthetic androgen. In certain aspects an androgen can include, but is not limited to R1881, testosterone, testosterone undecanoate, testosterone cypionate, testosterone enanthate, fluoxymesterone, methyltestosterone, 5α-dihydrotestosterone (DHT), or other known natural or synthetic androgens. A number of therapeutic androgens are known in the art.

Calcitriol or its analog and an androgen can be formulated individually and administered in a regime that combines the agents or calcitriol or its analog and an androgen can be co-formulated to form a therapeutic composition. A pharmaceutical composition, as used herein, refers to a mixture of one or more therapeutic agents described herein (e.g., calcitriol and/or an androgen) with other chemical components, such as carriers, stabilizers, diluents, dispersing agents, suspending agents, thickening agents, and/or excipients. The pharmaceutical composition facilitates administration of one or more agents to a subject. Pharmaceutical composition containing one or more agents can be administered in therapeutically effective amounts as pharmaceutical compositions by any conventional form and route known in the art including, but not limited to: intravenous, oral, rectal, aerosol, parenteral, pulmonary, transdermal, nasal, and topical administration.

One may administer the one or more agents in a local rather than systemic manner, for example, via injection of the compound directly into an organ such as the prostate, often in a depot or sustained release formulation. In addition, the pharmaceutical composition containing one or more agents may be provided in the form of a rapid release formulation, in the form of an extended release formulation, and/or in the form of an intermediate release formulation.

For oral administration, one or more agents can be formulated readily by combining the active compounds with pharmaceutically acceptable carriers or excipients well known in the art. Such carriers enable the compounds described herein to be formulated as tablets, powders, pills, dragees, capsules, liquids, gels, syrups, elixirs, slurries, suspensions and the like, for oral ingestion by a subject to be treated.

Pharmaceutical preparations which can be used orally include push-fit capsules made of gelatin, as well as soft, sealed capsules made of gelatin and a plasticizer, such as glycerol or sorbitol. In some embodiments, the capsule comprises a hard gelatin capsule comprising one or more of pharmaceutical, bovine, and plant gelatins. In certain instances, a gelatin is alkaline processed. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal or sublingual administration, the compositions may take the form of tablets, lozenges, or gels formulated in conventional manner. Parental injections may involve for bolus injection or continuous infusion. The pharmaceutical composition of one or more agents may be in a form suitable for parenteral injection as a sterile suspensions, solutions or emulsions in oily or aqueous vehicles, and may contain excipients such as suspending, stabilizing and/or dispersing agents. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions. Alternatively, the active ingredient may be in powder form for constitution with a suitable vehicle, e.g., sterile pyrogen-free water, before use.

One or more agents can be administered topically and can be formulated into a variety of topically administrable compositions, such as solutions, suspensions, lotions, gels, pastes, medicated sticks, balms, creams or ointments. Such pharmaceutical composition can contain solubilizers, stabilizers, tonicity enhancing agents, buffers and preservatives.

For administration by inhalation, one or more agents may be in a form as an aerosol, a mist or a powder. Pharmaceutical compositions are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, such as, by way of example only, gelatin for use in an inhaler or insufflator may be formulated containing a powder mix of the compound and a suitable powder base such as lactose or starch.

In practicing the methods of treatment or use provided herein, therapeutically effective amounts of one or more agents provided herein are administered in a pharmaceutical composition to a mammal having a prostate disease or condition to be treated. In some embodiments, the mammal is a human. A therapeutically effective amount can vary widely depending on the severity of the disease, the age and relative health of the subject, the potency of the compound used and other factors. The compounds can be used singly or in combination with one or more therapeutic agents as components of mixtures. In certain aspects calcitriol or analog thereof and one or more androgen are used in combination. In a further aspect calcitriol or analog thereof and one or more androgen are co-formulated to form a therapeutic composition.

Pharmaceutical compositions may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art. Pharmaceutical compositions described herein may be manufactured in a conventional manner, such as, by way of example only, by means of conventional mixing, dissolving, granulating, dragee-making, levigating, emulsifying, encapsulating, entrapping or compression processes.

The pharmaceutical compositions can include at least one pharmaceutically acceptable carrier, diluent or excipient and one or more agents described herein as an active ingredient in free-base form, or in a pharmaceutically acceptable salt form.

Methods for the preparation of compositions comprising one or more agents described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The compositions may be in liquid solutions or suspensions, solid forms suitable for solution or suspension in a liquid prior to use, or as emulsions. These compositions may also contain minor amounts of nontoxic, auxiliary substances, such as wetting or emulsifying agents, pH buffering agents, and so forth.

A summary of types of pharmaceutical compositions may be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference herein in its entirety.

Therapeutic agents or compositions described herein can be used in the preparation of medicaments for the treatment of diseases or conditions of the prostate. In addition, a method for treating any of the diseases or conditions described herein in a subject in need of such treatment, involves administration of pharmaceutical compositions containing at least one compound of the agents described herein or a pharmaceutically acceptable salt, pharmaceutically-acceptable prodrug, or pharmaceutically acceptable solvate thereof, in therapeutically-effective amounts to said subject.

The compositions containing the compound(s) described herein can be administered for prophylactic and/or therapeutic treatments. In therapeutic applications, the compositions are administered to a subject already suffering from a disease or condition, in an amount sufficient to treat or at least partially arrest the symptoms of the disease or condition, or to heal, improve, or ameliorate the condition itself. Amounts effective for this use will depend on the severity and course of the disease or condition, previous therapy, the subject's health status, weight, and response to the drugs, and the judgment of the treating physician.

Once improvement of the subject's conditions has occurred, a maintenance dose is administered if necessary. Subsequently, the dosage or the frequency of administration, or both, can be reduced, as a function of the symptoms, to a level at which the improved disease or condition is retained. Subjects can, however, require intermittent treatment on a long-term basis upon any recurrence of symptoms.

In certain instances, it may be appropriate to administer therapeutically effective amounts of at least one of the compounds described herein (or a pharmaceutically acceptable salts, pharmaceutically active metabolites, pharmaceutically-acceptable prodrugs, and pharmaceutically acceptable solvates thereof) in combination with another therapeutic agent. By way of example only, if one of the side effects experienced by a subject upon receiving one of the compounds herein is inflammation, then it may be appropriate to administer an anti-inflammatory agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the subject is enhanced). Or, by way of example only, the benefit of experienced by a subject may be increased by administering one of the compounds described herein with another therapeutic agent (which also includes a therapeutic regimen) that also has therapeutic benefit. In any case, regardless of the disease or condition being treated, the overall benefit experienced by the subject may simply be additive of the two therapeutic agents or the subject may experience a synergistic benefit. Where the compounds described herein are administered in conjunction with other therapies, dosages of the co-administered compounds will of course vary depending on the type of co-drug employed, on the specific drug employed, on the disease or condition being treated and so forth. In addition, when co-administered with one or more biologically active agents, the compound provided herein may be administered either simultaneously with the biologically active agent(s), or sequentially. If administered sequentially, the attending physician will decide on the appropriate sequence of administering protein in combination with the biologically active agent(s).

In any case, the multiple therapeutic agents (one of which is one of the compounds described herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may vary from more than zero weeks to less than four weeks. In addition, the combination methods, compositions and formulations are not to be limited to the use of only two agents. Multiple therapeutic combinations are envisioned.

The combination therapies described herein can be administered before, during or after the occurrence of a disease or condition, and the timing of administering the composition containing a therapeutic agent can vary. Thus, for example, the compounds can be used as a prophylactic and can be administered continuously to subjects with a propensity to conditions or diseases in order to prevent the occurrence of the disease or condition. The compounds and compositions can be administered to a subject during or as soon as possible after the onset of the symptoms. The initial administration can be via any route practical, such as, for example, an intravenous injection, a bolus injection, infusion over 5 minutes to about 5 hours, a pill, a capsule, transdermal patch, buccal delivery, and the like, or combination thereof. An agent is preferably administered as soon as is practicable after the onset of a disease or condition is detected or suspected, and for a length of time necessary for the treatment of the disease, such as, for example, from about 1 month to about 3 months. The length of treatment can vary for each subject, and the length can be determined using the known criteria. For example, the therapeutic agent(s) or a formulation containing the same can be administered for at least 2 weeks, preferably about 1 month to about 3 years and in some embodiments from about 1 month to about 10 years. In other embodiments, the compound is administered once a day from 90 days to 2 years.

The pharmaceutical composition described herein may be in unit dosage forms suitable for single administration of precise dosages. In unit dosage form, the formulation is divided into unit doses containing appropriate quantities of one or more therapeutic agents. The unit dosage may be in the form of a package containing discrete quantities of the formulation. Non-limiting examples are packaged tablets or capsules, and powders in vials or ampules.

The daily dosages appropriate for calcitriol or analog thereof and/or androgens described herein are from about 0.03 to 100 mg/kg per body weight. An indicated daily dosage in a larger mammal, including, but not limited to, humans, is in the range from about 1 mg to about 4000 mg, conveniently administered in one or more doses, including, but not limited to, up to four times a day or in retard form. Suitable unit dosage forms for oral administration comprise from about 1 mg to about 4000 mg active ingredient. In some embodiments, a single dose of agent(s) is within the range of about 50 mg to about 2,000 mg. In some embodiments, a single dose of agent(s) is about 90 mg, about 200 mg, about 250 mg, about 325 mg, about 650 mg, about 975 mg, about 1300 mg, about 1625 mg, or about 1950 mg. In some embodiments, an administration of one or more agents of about 90 mg, about 325 mg, about 650 mg, about 975 mg, about 1300 mg, about 1625 mg, or about 1950 mg is given as multiple doses.

In some embodiments, the single dose of one or both therapeutic agent is between 90 to 2500 mgs and the one or more therapeutic agent is administered to a subject for between 90 days to two years.

Such dosages may be altered depending on a number of variables, not limited to the activity of the compound used, the disease or condition to be treated, the mode of administration, the requirements of the individual subject, the severity of the disease or condition being treated, and the judgment of the practitioner.

1. A method of reducing 3-β hydroxysteroid dehydrogenase-type I (HSD3β-1) levels comprising administering vitamin D and an androgen to a subject in need thereof.

2. The method of claim 1, wherein vitamin D is calcitriol or an analog thereof.

3. The method of claim 1, wherein the androgen is testosterone or an analog thereof

4. The method of claim 1, wherein the vitamin D is administered about 1, 2, 3, 4, 5, 6, 7, 8, 9, or 10, hours or days prior to administration of the androgen.

5. The method of claim 1, wherein the androgen is administered about 1, 2, 3, 4, 5, 6, 7, 8, 9, or 10, hours or days prior to administration of the vitamin D.

6. The method of claim 1, wherein the vitamin D and androgen are administered within about 1, 2, 3, 4, 5, 6, 7, 8, 9, 10 minutes or days of each other.

7. The method of claim 1, wherein the vitamin D and androgen are administered simultaneously.

8. The method of claim 1, wherein the vitamin D and androgen are formulated in the same composition (co-formulated).

9. A method of treating metastatic castration-resistant prostate cancer (mCRPC) comprising administering vitamin D and an androgen to a subject having mCRPC.

10. The method of claim 1, wherein vitamin D is calcitriol or an analog thereof.

11. The method of claim 1, wherein the androgen is testosterone or an analog thereof

12. The method of claim 1, wherein the vitamin D is administered about 1, 2, 3, 4, 5, 6, 7, 8, 9, or 10, hours or days prior to administration of the androgen.

13. The method of claim 1, wherein the androgen is administered about 1, 2, 3, 4, 5, 6, 7, 8, 9, or 10, hours or days prior to administration of the vitamin D.

14. The method of claim 1, wherein the vitamin D and androgen are administered within about 1, 2, 3, 4, 5, 6, 7, 8, 9, 10 minutes or days of each other.

15. The method of claim 1, wherein the vitamin D and androgen are administered simultaneously.

16. The method of claim 1, wherein the vitamin D and androgen are formulated in the same composition (co-formulated).

17. A composition comprising a vitamin D compound and androgen.

18. The composition of claim 17, wherein the vitamin D compound is calcitriol or EB1089.

19. The composition of claim 17, wherein the androgen is testosterone or R1881.
